(19) United States
(12) Patent Application Publication
(10) Pub. No.: US 2009/0053193 A1
(43) Pub. Date: Feb. 26, 2009

(54) Use of Factor VIIa for the Treatment of Burn Trauma

(75) Inventors: Par Johannson, Dosjebro (SE); Rasmus Rojkjaer, Princeton, NJ (US)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk HealthCare A/G, Zurich (CH)

(21) Appl. No.: 11/579,680

(22) PCT Filed: May 11, 2005
(86) PCT No.: PCT/EP2005/052150

§ 371 Date: Oct. 24, 2008

Related U.S. Application Data
(60) Provisional application No. 60/569,867, filed on May 11, 2004.

(51) Int. Cl. A61K 38/48; C12N 9/50

(52) U.S. Cl. 424/94.64; 435/212

(57) ABSTRACT

The invention relates to the use of Factor VIIa or a Factor VIIa equivalent for the manufacture of a medicament for treatment of burn trauma.

### FIELD OF THE INVENTION

The present invention relates to methods for acute treatment of burn traumas, including the prevention of, or minimizing severity of, late complications in burned trauma patients.

### BACKGROUND OF THE INVENTION

Haemostasis is a complex physiological process which ultimately results in the arrest of bleeding. This is dependent on the proper function of three main components: blood vessels (especially the endothelial lining), coagulation factors, and platelets. Once a haemostatic plug is formed, the timely activation of the fibrinolytic system is equally important to prevent further unnecessary haemostatic activation. Any malfunction of this system (due to a reduced number, or molecular dysfunction, of the haemostatic components or increased activation of the fibrinolytic components) may lead to clinical bleeding such as, e.g., haemorrhagic diathesis of varying severity.

In most physiological situations, haemostasis is triggered by the interaction of circulating activated coagulation factor VII (FVIIa) with tissue factor (TF) subsequent to exposure of TF at the site of an injury. Endogenous FVIIa becomes proteolytically active only after forming a complex with TF. Normally, TF is expressed in the deep layers of the vessel wall and is exposed following injury. This ensures a highly localized activation of coagulation and prevents disseminated coagulation. TF also seems to exist in a non-active form, so-called encrypted TF. The regulation of encrypted versus active TF is still unknown.

Activated recombinant human factor VII (rFVIIa) is indicated for the treatment of bleeding episodes in haemophilia A or B subjects with inhibitors to Factor VIII or Factor IX. When given in high (pharmacological) doses, rFVIIa can bind independently of TF to activated platelets and initiate local thrombin generation which is important for the formation of the initial haemostatic plug.

Patients suffering from burn trauma often require surgery and/or experience microvascular and other bleeding.

Thus, there is a need in the art for improved methods and compositions for acute treatment of burn trauma, as well as for prevention and attenuation of late complications that result from burn trauma.

### SUMMARY OF THE INVENTION

The invention provides the use of Factor VIIa or a Factor VIIa equivalent for the manufacture of a medicament for treatment of burn trauma. Typical patients for whom the medicament is used are those patients who have experienced extensive burn trauma, including, without limitation, those suffering from coagulopathic bleedings.

The invention also provides methods for treating burn trauma, which are carried out by administering to a patient an effective amount for said preventing or attenuating of Factor VIIa or a Factor VIIa equivalent. Typical patients having experienced burn trauma need an excision of tissue, such as an excision of tissue of 5% or more of TBSA, such as 10 or more of TBSA. Typical patients also experience microvascular bleedings.

Excision of burn wounds is frequently associated with a large volume of blood loss requiring allogeneic blood transfusion. Thus in some embodiments the administration of Factor VIIa or a Factor VIIa equivalent reduces blood transfusion requirement in burn patients undergoing excision and skin grafting.

In some embodiments, the initial administering step is carried out within 5 hours of the occurrence of the traumatic burn. In some embodiments, the initial administering step is carried out immediately before start of excision surgery. In some embodiments, an administering step is carried out after excision surgery, such as repeated 60 minutes after. In some embodiments, the effective amount comprises at least about 40 μg/kg, such as at least about 80 μg/kg of Factor VIIa or a corresponding amount of a Factor VIIa equivalent. In some embodiments, the effective amount comprises at least about 100 μg/kg of Factor VIIa or a corresponding amount of a Factor VIIa equivalent. In some embodiments, a first amount of at least about 40 μg/kg, such as at least about 80 μg/kg Factor VIIa or a corresponding amount of a Factor VIIa equivalent is administered at the start of treatment, and a second amount of at least about 40 μg/kg, such as at least about 80 μg/kg of Factor VIIa or a corresponding amount of a Factor VIIa equivalent is administered to the patient one or more hours after the start of treatment. In some embodiments, a third amount of at least about 40 μg/kg, such as at least about 80 μg/kg of Factor VIIa or a corresponding Factor VIIa equivalent is administered at a later time, such as, e.g. least about one hour after the start of the second treatment.

In some embodiments, the method further comprises administering to the patient a second coagulation agent in an amount that augments the treatment by said Factor VIIa or Factor VIIa equivalent. Preferably, the second coagulation agent is a coagulation factor (including, without limitation, Factor V, Factor VIII, Factor IX, Factor X, Factor XI, Factor XIII, Fibrinogen, thrombin, TAFI; an antifibrinolytics such as, e.g., PAI-1, aprotinin, epsilon-aminocaproic acid or tranexamic acid, various antithrombotic treatments, as well as transfusions with platelet, RBC, FFP, oxygen carriers, the various bypassing agents and fluid therapies (colloids/crystalloids), or any combination thereof.

The present invention also provides a kit of parts for treatment of burn trauma, comprising

(i) A medicament comprising Factor VIIa or a Factor VIIa equivalent; and
(ii) Instructions for use describing that:
a. A first dose containing at least about 40 μg/kg, such as at least about 80, such as at least about 100 μg/kg Factor VIIa or a corresponding amount of a Factor VIIa equivalent, should be administered at the start of treatment;
b. A second dose containing at least about 40 μg/kg, such as at least about 80 μg/kg Factor VIIa or a corresponding amount of a Factor VIIa equivalent should be administered one to 24 hours after the start of treatment.

The present invention also provides methods for treating burn trauma in burn trauma patients, which are carried out by: (i) administering to a group of burn trauma patients an amount effective for treatment of Factor VIIa or a Factor VIIa equivalent; and (ii) observing a reduction in late complications of burn trauma among the group of patients who received Factor VIIa or a Factor VIIa equivalent relative to the frequency of occurrence of said late complications that would have been expected in the same group of patients who had not received said Factor VIIa or Factor VIIa equivalent.

### DETAILED DESCRIPTION OF THE INVENTION

Severe burn injury leads to an inflammatory response, suppression of cellular immune response and disturbances of the haemostatic system. These pathological changes are most pronounced during the first week post-injury and may be further enhanced by surgery.

Typically, patients having experienced burn trauma need an excision of tissue and/or experience microvascular bleedings, i.e. oozing bleedings, such as oozing continuous bleedings from mucosal surfaces.

Excision of burn wounds is usually associated with large volume blood loss caused by consumption coagulopathy, haemodilution and the surgical techniques used, requiring allogeneic blood transfusion. Blood transfusion is associated with a number of infectious and non-infectious complications. Furthermore, in burn patients, transfusion of allogeneic blood may further suppress cellular immune response resulting in increased infectious morbidity.

In a first aspect the present invention relates to the use of Factor VIIa or a Factor VIIa equivalent for the manufacture of a medicament for treating burn trauma.

In a second aspect the present invention relates to a kit of parts for treatment of burn trauma, comprising

(i) A medicament comprising Factor VIIa or a Factor VIIa equivalent; and
(ii) Instructions for Use describing that:
a. A first dose containing at least about 40 μg/kg, such as at least about 80, such as at least about 100 μg/kg Factor VIIa or a corresponding amount of a Factor VIIa equivalent, should be administered at the start of treatment;
b. A second dose containing at least about 40 μg/kg, such as at least about 80 μg/kg Factor VIIa or a corresponding amount of a Factor VIIa equivalent should be administered one to 24 hours after the start of treatment.

In a third aspect the present invention relates to a method for treating burn trauma, the method comprising administering to a patient in need of said treatment an effective amount for said treatment of Factor VIIa or a Factor VIIa equivalent.

In a further aspect the present invention relates to a method for preventing treating burn trauma, the method comprising intentionally administering to a patient in need of said treatment an effective amount for said treatment of Factor VIIa or a Factor VIIa equivalent for the purpose of treating burn trauma.

In a further aspect the present invention relates to a method for treating burn trauma in burn trauma patients, said method comprising (i) administering to a group of burn trauma patients an effective amount for said treatment of Factor VIIa or a Factor VIIa equivalent; and (ii) observing a reduction in one or more clinical parameters of burn trauma among said group of patients relative to the level of said clinical parameters that would have been expected in the same group of patients who had not received said Factor VIIa or Factor VIIa equivalent.

In a further aspect the present invention relates to a method of reducing the amount of microvascular bleeding the method comprising administering to a patient in need of said treatment an effective amount for said treatment of Factor VIIa or a Factor VIIa equivalent.

In a further aspect the present invention relates to a method of increasing the likelihood of tissue graft survival the method comprising administering to a patient in need of said treatment an effective amount for said treatment of Factor VIIa or a Factor VIIa equivalent.

In a further aspect the present invention relates to a method of increasing the time of tissue graft survival the method comprising administering to a patient in need of said treatment an effective amount for said treatment of Factor VIIa or a Factor VIIa equivalent.

In a further aspect the present invention relates to a method of decreasing or modulating the inflammatory response to surgery-induced tissue injury the method comprising administering to a patient in need of said treatment an effective amount for said treatment of Factor VIIa or a Factor VIIa equivalent.

One aspect of the present invention relates to the use of Factor VIIa or a Factor VIIa equivalent in reducing perioperative blood transfusion requirement in burn patients undergoing excision and skin grafting. In one series of embodiments, treatment of patients according to the invention results in reduction in a perioperative blood transfusion requirement by 10%, such as 20%, such as 40%, such as 60%, such as 80%, such as 100%.

Burn wound excision and skin grafting is a standard treatment for patients with severe burn injury. The optimal timing for surgery after initial burn injury remains a matter for debate albeit most centres perform surgery within 48 hours post injury and prefer a multi-stage approach. However, since the levels of inflammatory cytokines are highest immediately after injury and burn injury leads to complex changes in the haemostatic system and induction of hypercoagulable state, skin excision and skin grafting are preferably carried out 4 days after the burn or later. This approach allows patients to be haemodynamically stabilised after the initial resuscitation phase, hence excision and grafting can be carried out in a one-stage surgery regardless of the size of wound excised and grafted.

Surgical excision and skin grafting are associated with excessive perioperative bleeding. The causes of bleeding are multifactorial including consumption coagulopathy, haemodilution, and the surgical technique used. Allogeneic blood transfusion is frequently needed in patients undergoing excision and skin grafting. Nevertheless, the immunomodulatory effect of allogeneic blood may worsen postoperative outcome.

In patients with extensive burn traumas (>10%) of total body surface area (TBSA) coagulopathy is often seen. This is induced both by the massive inflammatory response at the site of termic traumatisation as well as by the later surgery during excision of damaged tissue and grafting of new tissue often applied. Initially during excision the patients have bleeding tendencies caused by dilution of coagulation factors and also by consumption of coagulation factors at the site of injury, in the necrotic coagulation active tissue.

The time for excision of damaged or necrotic tissue at the site of burn trauma varies from 1 day to 14 days post trauma. Some patients, usually with very extensive burns, may have developing sepsis and inflammation, which may require early excision, usually at day 4-5 after the trauma. Increased bleeding is expected at early excision.

The present invention is particular suitable for severely burned patients (needing an excision of tissue of 10% or more of TBSA) because the have (i) a substantial need for per- and postoperative transfusions, (ii) bleeding severity is correlated to patient morbidity and ultimately mortality, (iii) the patients are at high risk of developing microvascular bleeding further increasing transfusion requirements and negatively affecting morbidity and mortality, and/or (iiii) the patients need surgical techniques that could induce massive sub endothelial TF exposure, which may also lead to a high risk patient population, which has an abundance of disseminated TF expression.

The present invention provides methods and compositions that can be used advantageously to treat burn trauma patients. The methods are carried out by administering to a burn trauma patient Factor VIIa or a Factor VIIa equivalent, in a manner that is effective for treatment. A manner effective for treatment may comprise administering a predetermined amount of Factor VIIa or a Factor VIIa equivalent, and/or utilizing a particular dosage regimen, formulation, mode of administration, combination with other treatments, and the like. The efficacy of the methods of the invention in treating burn trauma may be assessed using one or more conventionally used parameters of the immediate consequences of injury and/or late complications. Immediate consequences include, e.g., blood loss and symptoms of shock; while late complications, include, without limitation, Pulmonary embolism (PE), Acute Respiratory Distress Syndrome (ARDS), Disseminated Intravascular Coagulation (DIC), Acute Myocardial Infarction (AMI), Cerebral Thrombosis (CT), Systemic Inflammatory Response Syndrome (SIRS), infections, sepsis, Multiple Organ Failure (MOF), and Acute Lung Injury (ALI), including death caused by one or more of these syndromes.

Thus in some aspects the present invention relates to a method of reducing the risk of immediate consequences of injury and/or late complications the method comprising administering to a patient in need of said treatment an effective amount for said treatment of Factor VIIa or a Factor VIIa equivalent. In one embodiment the present invention relates to a method of reducing the risk of immediate consequences of injury and/or late complications selected from the list consisting of Pulmonary embolism (PE), Acute Respiratory Distress Syndrome (ARDS), Disseminated Intravascular Coagulation (DIC), Acute Myocardial Infarction (AMI), Cerebral Thrombosis (CT), Systemic Inflammatory Response Syndrome (SIRS), infections, sepsis, Multiple Organ Failure (MOF), and Acute Lung Injury (ALI), including death caused by one or more of these syndromes, the method comprising administering to a patient in need of said treatment an effective amount for said treatment of Factor VIIa or a Factor VIIa equivalent. In one embodiment the late complication is Multiple Organ Failure (MOF). In one embodiment the late complication is Acute Respiratory Distress Syndrome (ARDS). In one embodiment the late complication is Acute Lung Injury (ALI). In one embodiment the late complication is sepsis.

Coagulopathy in trauma is multifactorial, encompassing coagulation abnormalities resembling DIC, caused by systemic activation of coagulation and fibrinolysis; excessive fibrinolysis, which can be evident on the first day in some trauma subjects; and dilutional coagulopathy, which is caused by excessive fluid administration. Some fluids such as hydroxyethyl starch (HES) preparations may directly compromise coagulation. Massive transfusion syndrome results in depletion of coagulation factors and impairment of platelet function. Hypothermia causes a slower enzyme activity of the coagulation cascade and dysfunctional platelets. Metabolic abnormalities, such as acidosis, also compromise coagulation especially when associated with hypothermia.

Non-limiting examples of patients in need of treatment according to the invention include those who exhibit one or more of the following:
Coagulation abnormalities resembling DIC, caused by systemic activation of coagulation and fibrinolysis
Excessive fibrinolysis
Dilutional coagulopathy caused by excessive fluid treatment, including, without limitation, a limited number of platelets and/or an impaired platelet function compared to the platelet count and platelet activity of normal pooled blood
Receipt of hydroxyethyl starch (HES) preparations
Hypothermia, a including having body temperature below about 37° C., such as, e.g., below about 36° C., below about 35° C., or below about 34° C.
At least one indication of metabolic abnormalities, including, without limitation, acidosis having a blood pH below about 7.5, such as, e.g., below about 7.4, below about 7.3, below about 7.2, or below about 7.1.

The methods of the present invention can be applied advantageously to any patient who has suffered burn trauma that and/or has required excision of burned tissue, if left untreated, would result in a significant loss of blood, such as, e.g., over 10% of the patient's total blood volume (loss of over 40% of blood volume is immediately lifethreatening.) A normal blood volume represents about 7% of an adult's ideal body weight and about 8-9% of a child's ideal body weight.

In one series of embodiments, patients treated according to the invention are those who are suffering from burn trauma needing an excision of tissue of 10% or more of TBSA. In another series of embodiments, patients treated according to the invention are those who are suffering from burn trauma needing an excision of tissue of 15% or more of TBSA. In another series of embodiments, patients treated according to the invention are those who are suffering from burn trauma needing an excision of tissue of 20% or more of TBSA. In another series of embodiments, patients treated according to the invention are those who are suffering from burn trauma needing an excision of tissue of 25% or more of TBSA. In another series of embodiments, patients treated according to the invention are those who are suffering from burn trauma needing an excision of tissue of 30% or more of TBSA. In another series of embodiments, patients treated according to the invention are those who have microvascular bleedings. In another series of embodiments, patients needing an excision of tissue and treated according to the invention have prolonged skin graft survival. It is to be understood that the skin grafts of patients treated according to the invention may survive for longer time and without further intervention than in the absence of treatment according to the invention. Thus the treatment according to the present invention provides a long-term graft maintenance and no or to a lesser extend graft rejection following tissue grafting in burn trauma patients.

In one series of embodiments, patients treated according to the invention are those who require transfusion with whole blood (WB), packed red blood cells (pRBC), or fresh frozen plasma (FFP), such as, e.g., more than about 2 units, 5 units, or more than about 8 units, between the time of their traumatic burn injury and the time of administration of Factor VIIa or Factor VIIa equivalent. A unit of WB typically contains about 450 ml blood and 63 ml of conventional anticoagulant/preservative (having a hematocrit of 36-44%). A unit of PRBC typically contains 200-250 ml of red blood cells, plasma, and conventional anticoagulant/preservative (having a hematocrit of 70-80%). In one series of embodiments, patients treated according to the invention do not suffer from a bleeding disorder, whether congential or acquired, such as, e.g., Hemophilia A, B. or C.

In different embodiments of the invention, patients may be excluded from treatment if they have received transfusion of 10 units or more of PRBC, such as, e.g., more than 15, 20, 25, or 30 units, or if they have been diagnosed with a congenital bleeding disorder, or if they have inhalation injury.

Factor VIIa and Factor VIIa Equivalents:

In practicing the present invention, any Factor VIIa or Factor VIIa equivalent may be used that is effective in treating a burn trauma. In some embodiments, the Factor VIIa is human Factor VIIa, as disclosed, e.g., in U.S. Pat. No. 4,784,950 (wild-type Factor VII).

The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa.

As used herein, “wild type human FVIIa” is a polypeptide having the amino acid sequence disclosed in U.S. Pat. No. 4,784,950.

Factor VIIa equivalents include, without limitation, Factor VII polypeptides that have either been chemically modified relative to human Factor VIIa and/or contain one or more amino acid sequence alterations relative to human Factor VIIa. Such equivalents may exhibit different properties relative to human Factor VIIa, including stability, phospholipid binding, altered specific activity, and the like. This includes FVII variants, Factor VII-related polypeptides, Factor VII derivatives and Factor VII conjugates exhibiting substantially the same or improved biological activity relative to wild-type human Factor VIIa.

The term “Factor VII derivative” as used herein, is intended to designate a FVII polypeptide exhibiting substantially the same or improved biological activity relative to wild-type Factor VII, in which one or more of the amino acids of the parent peptide have been genetically and/or chemically and/or enzymatically modified, e.g. by alkylation, glycosylation, PEGylation, acylation, ester formation or amide formation or the like. This includes but is not limited to PEGylated human Factor VIIa, cysteine-PEGylated human Factor VIIa and variants thereof.

The term “improved biological activity” refers to FVII polypeptides with i) substantially the same or increased proteolytic activity compared to recombinant wild type human Factor VIIa or ii) to FVII polypeptides with substantially the same or increased TF binding activity compared to recombinant wild type human Factor VIIa or iii) to FVII polypeptides with substantially the same or increased half life in blood plasma compared to recombinant wild type human Factor VIIa. The term “PEGylated human Factor VIIa” means human Factor VIIa, having a PEG molecule conjugated to a human Factor VIIa polypeptide. It is to be understood, that the PEG molecule may be attached to any part of the Factor VIIa polypeptide including any amino acid residue or carbohydrate moiety of the Factor VIIa polypeptide. The term “cysteine-PEGylated human Factor VIIa” means Factor VIIa having a PEG molecule conjugated to a sulfhydryl group of a cysteine introduced in human Factor VIIa.

In one series of embodiments, a Factor VIIa equivalent includes polypeptides that exhibit at least about 10%, preferably at least about 30%, more preferably at least about 50%, and most preferably at least about 70%, of the specific biological activity of human Factor VIIa. For purposes of the invention, Factor VIIa biological activity may be quantified by measuring the ability of a preparation to promote blood clotting using Factor VII-deficient plasma and thromboplastin, as described, e.g., in U.S. Pat. No. 5,997,864. In this assay, biological activity is expressed as the reduction in clotting time relative to a control sample and is converted to “Factor VII units” by comparison with a pooled human serum standard containing 1 unit/ml Factor VII activity. Alternatively, Factor VIIa biological activity may be quantified by (i) measuring the ability of Factor VIIa or a Factor VIIa equivalent to produce of Factor Xa in a system comprising TF embedded in a lipid membrane and Factor X. (Persson et al., J. Biol. Chem. 272:19919-19924, 1997); (ii) measuring Factor X hydrolysis in an aqueous system (see, Example 5 below); (iii) measuring the physical binding of Factor VIIa or a Factor VIIa equivalent to TF using an instrument based on surface plasmon resonance (Persson, FEBS Letts. 413:359-363, 1997) and (iv) measuring hydrolysis of a synthetic substrate by Factor VIIa and/or a Factor VIIa equivalent.

Examples of factor VII equivalents include, without limitation, wild-type human Factor VIIa, L305V-FVII, L305V/M306D/D309S-FVII, L305I-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L30SV/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158 D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A —FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A —FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M 298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M 298Q-FVII, F374Y/E296V/M298Q-FVII, F374Y/L305V/K337A/V158 D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M 298Q/K337A/5314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/5314E-FVII, F374Y/L305V/V158D/E296V/M298Q/5314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/5314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R152E-Factor VII, S344A-Factor VII, Factor VIIa lacking the Gla domain; and P11QK33E-FVII, T106N-FVII, K143N/N145T-FVII, V253N-FVII, R290N/A292T-FVII, G291N-FVII, R315N/V317T-FVII, K143N/N145T/R315N/V317T-FVII; and FVII having substitutions, additions or deletions in the amino acid sequence from 233Thr to 240Asn, FVII having substitutions, additions or deletions in the amino acid sequence from 304Arg to 329Cys.

Other examples of factor VII equivalents include, without limitation Factor VII equivalents having substantially the same biological activity as wild-type Factor VII including S52A-FVIIa, S60A-FVIIa (Lino et al., Arch. Biochem. Biophys. 352: 182-192, 1998); FVIIa equivalents exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII equivalents as disclosed in WO 02/29025; and FVII equivalents exhibiting increased proteolytic stability as disclosed in WO 02/38162 (Scripps Research Institute); FVII equivalents having a modified Gla-domain and exhibiting an enhanced membrane binding as disclosed in WO 99/20767, WO 00/66753, WO 02/02764, and US patent application 20030211094 (University of Minnesota); and FVII equivalents as disclosed in WO 01/04287, WO 01/58935, WO 03/93465, and US patent application 20030165996 (Maxygen ApS).

Other examples of factor VII equivalents include GlycoPegylated FVII derivatives as disclosed in WO 03/31464 and US Patent applications US 20040043446, US 20040063911, US 20040142856, US 20040137557, and US 20040132640 (Neose Technologies, Inc.).

Non-limiting examples of FVII equivalents having increased biological activity compared to wild-type FVIIa include FVII equivalents as disclosed in WO 01/83725, WO 02/22776, WO 02/077218, WO 03/027147, WO 04/029090, WO 04/000366, WO 03/037932; Danish patent application PA 2003 01296, Danish patent application PA 2004 00160, Danish patent application PA 2003 01145, WO 02/38162 (Scripps Research Institute); and FVIIa equivalents with enhanced activity as disclosed in JP 2001061479 (Chemo-Sero-Therapeutic Res Inst.).

Preparations and Formulations:

The present invention encompasses therapeutic administration of Factor VIIa or Factor VIIa equivalents, which is achieved using formulations that comprise Factor VIIa preparations. As used herein, a “Factor VII preparation” refers to a plurality of Factor VIIa polypeptides or Factor VIIa equivalent polypeptides, including variants and chemically modified forms, that have been separated from the cell in which they were synthesized, whether a cell of origin or a recombinant cell that has been programmed to synthesize Factor VIIa or a Factor VIIa equivalent.

Separation of polypeptides from their cell of origin may be achieved by any method known in the art, including, without limitation, removal of cell culture medium containing the desired product from an adherent cell culture; centrifugation or filtration to remove non-adherent cells; and the like.

Optionally, Factor VII polypeptides may be further purified. Purification may be achieved using any method known in the art, including, without limitation, affinity chromatography, such as, e.g., on an anti-Factor VII antibody column (see, e.g., Wakabayashi et al., J. Biol. Chem. 261:11097, 1986; and Thim et al., Biochem. 27:7785, 1988); hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J.-C. Janson and Lars Ryden, editors, VCH Publishers, New York, 1989. Following purification, the preparation preferably contains less than about 10% by weight, more preferably less than about 5% and most preferably less than about 1%, of non-Factor VII proteins derived from the host cell.

Factor VII and Factor VII-related polypeptides may be activated by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat. No. 4,456,591; and Hedner et al., J. Clin. Invest. 71:1836 (1983). Alternatively, Factor VII may be activated by passing it through an ion-exchange chromatography column, such as Mono Q® (Pharmacia) or the like. The resulting activated Factor VII may then be formulated and administered as described below.

Pharmaceutical compositions or formulations for use in the present invention comprise a Factor VIIa preparation in combination with, preferably dissolved in, a pharmaceutically acceptable carrier, preferably an aqueous carrier or diluent. A variety of aqueous carriers may be used, such as water, buffered water, 0.4% saline, 0.3% glycine and the like. The preparations of the invention can also be formulated into liposome preparations for delivery or targeting to the sites of injury. Liposome preparations are generally described in, e.g., U.S. Pat. Nos. 4,837,028, 4,501,728, and 4,975,282. The compositions may be sterilised by conventional, well-known sterilisation techniques. The resulting aqueous solutions may be packaged for use or filtered under aseptic conditions and lyophilised, the lyophilised preparation being combined with a sterile aqueous solution prior to administration.

The compositions may contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc.

Treatment Regimen:

In practicing the present invention, Factor VIIa or the Factor VIIa equivalent may be administered to a patient as a single dose comprising a single-dose-effective amount for treating the burn trauma, or in a staged series of doses which together comprise an effective amount for treating burn trauma. An effective amount of Factor VIIa or the Factor VIIa equivalent (see below) refers to the amount of Factor VIIa or equivalent which, when administered in a single dose or in the aggregate of multiple doses, or as part of any other type of defined treatment regimen, produces a measurable improvement in at least one clinical parameter associated with burn trauma. When Factor VIIa equivalents are administered, an effective amount may be determined by comparing the coagulant activity of the Factor VIIa equivalent with that of Factor VIIa and adjusting the amount to be administered proportionately to the predetermined effective dose of Factor VIIa.

Administration of Factor VIIa or a Factor VIIa equivalent according to the present invention is preferably initiated within about 6 hours after occurrence of the traumatic burn injury, such as, e.g., within about 4 hours, within about 2 hours, or within about 1 hour. Alternatively, administration may be initiated at any time before start of surgery in patients in need of excision of burned tissue, such as within about 6 hours before surgery, such as, e.g., within about 4 hours, within about 2 hours, or within about 1 hour before surgery e.g., immediately before surgery.

Administration of a single dose refers to administration of an entire dose of Factor VIIa or the Factor VIIa equivalent as a bolus over a period of less than about 5 minutes. In some embodiments, the administration occurs over a period of less than about 2.5 minutes, and, in some, over less than about 1 min. Typically, a single-dose effective amount comprises at least about 40 μg/kg human Factor VIIa or a corresponding amount of a Factor VIIa equivalent, such as, at least about 50 μg/kg, 75 μg/kg, or 90 μg/kg, or at least 150 μg/kg Factor VIIa.

In some embodiments, following administration of a single dose of Factor VIIa or a Factor VIIa equivalent according to the invention, the patient receives no further Factor VIIa or Factor VIIa equivalent for an interval of at least about 30 minutes. In some embodiments the post-administration interval is at least about 45 minutes, such as at least about 1 hour, at least about 1.5 hours, or at least about 2 hours.

In other embodiments, the patient receives Factor VIIa or Factor VIIa equivalent according to the following regimen: (i) The patient receives a first amount of Factor VIIa or Factor VIIa equivalent comprising at least about 40 μg/kg; (ii) after a period of at least about 30 minutes, a second amount of Factor VIIa or Factor VIIa equivalent is administered, the amount comprising at least about 40 μg/kg; and (iii) after a period of at least about 30 minutes from administration of the second dose, a third amount of Factor VIIa or Factor VIIa equivalent is administered, the amount comprising at least about 40 μg/kg. After a period of at least about 30 minutes following the administration of the third amount, the patient may then receive a further (fourth) amount of Factor VIIa or Factor VIIa equivalent comprising at least about 40 μg/kg.

In other embodiments, the first amount of Factor VIIa or Factor VIIa equivalent comprises at least about 40 μg/kg, such as at least about 80 μg/kg, such as at least about 100 μg/kg or at least about 150 μg/kg; in other embodiments, the second amount of Factor VIIa or Factor VIIa equivalent comprises at least about 75 μg/kg, such as at least about 90 μg/kg; in other embodiments, the third (and optionally fourth) amount of Factor VIIa or Factor VIIa equivalent comprises at least about 75 μg/kg, such as at least about 90 μg/kg.

In one embodiment, the first dose comprises about 200 μg/kg, the second dose about 100 μg/kg, and the third (and optionally fourth) dose about 100 μg/kg.

In other embodiments, the patient receives the second amount of Factor VIIa or Factor VIIa equivalent after a period of at least about 45 minutes from the first administration, such as at least about 1 hour, at least about 1.5 hours, at least about 2 hours, at least about 2.5 hours, or at least about 3 hours.

In other embodiments, the patient receives the third (and optionally fourth) amount of Factor VIIa or Factor VIIa equivalent after a period of at least about 45 minutes from the previous administration, such as at least about 1 hour, at least about 1.5 hours, at least about 2 hours, at least about 2.5 hours, or at least about 3 hours.

In one embodiment, the patient receives a first dose comprising about 200 μg/kg; after a period of about 1 hour, the patient receives a second dose comprising about 100 μg/kg, and after a period of about 3 hours from the first dose, the patient receives a third dose comprising about 100 μg/kg.

The following table illustrates different non-limiting embodiments of the invention:

TABLE 1

| Time post- | | | | | | | | | | | | |
|---|---|---|---|---|---|---|---|---|---|---|---|---|
| injury or | | | | Time to | | | | Time to | | 3^{rd} Dose | | Additional |
| post excision | | 1^{st} Dose | | 2^{nd} dose | | 2^{nd} Dose (optional) | | 3^{rd} dose | | (optional) | | Doses |
| 0-≦1 | h | >40 | mcg/kg | 0-1 | h | >40 | mcg/kg | 0-1 | h | >40 | mcg/kg | As needed |
| | | | | 1-2 | h | | | 1-2 | h | | | |
| | | | | ≧3 | h | | | ≧3 | h | | | |
| | | 100 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |
| | | ≧150 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |
| >1-≦3 | h | >40 | mcg/kg | 0-1 | h | >40 | mcg/kg | 0-1 | h | >40 | mcg/kg | As needed |
| | | | | 1-2 | h | | | 1-2 | h | | | |
| | | | | ≧3 | h | | | ≧3 | h | | | |
| | | 100 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |
| | | ≧150 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |
| >3-<6 | h | >50 | mcg/kg | 0-1 | h | >50 | mcg/kg | 0-1 | h | >50 | mcg/kg | As needed |
| | | | | 1-2 | h | | | 1-2 | h | | | |
| | | | | ≧3 | h | | | ≧3 | h | | | |
| | | 100 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |
| | | ≧150 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |
| >6-<12 | h | >50 | mcg/kg | 0-1 | h | >50 | mcg/kg | 0-1 | h | >50 | mcg/kg | As needed |
| | | | | 1-2 | h | | | 1-2 | h | | | |
| | | | | ≧3 | h | | | ≧3 | h | | | |
| | | 100 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |
| | | ≧150 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |

It will be understood that the effective amount of Factor VIIa or Factor VIIa equivalent, as well as the overall dosage regimen, may vary according to the patient's haemostatic status, which, in turn, may be reflected in one or more clinical parameters, including, e.g., relative levels of circulating coagulation factors; amount of blood lost; rate of bleeding; hematocrit, and the like. It will be further understood that the effective amount may be determined by those of ordinary skill in the art by routine experimentation, by constructing a matrix of values and testing different points in the matrix.

For example, in one series of embodiments, the invention encompasses (i) administering a first dose of Factor VIIa or a Factor VIIa equivalent; (ii) assessing the patient's coagulation status after a predetermined time; and (iii) based on the assessment, administering a further dose of Factor VIIa or Factor VIIa equivalent if necessary. Steps (ii) and (iii) may be repeated until satisfactory hemostasis is achieved.

According to the invention, Factor VIIa or a Factor VIIa equivalent may be administered by any effective route, including, without limitation, intravenous, intramuscular, subcutaneous, mucosal, and pulmonary routes of administration. Preferably, administration is by an intravenous route.

### Combination Treatments:

The present invention encompasses combined administration of an additional agent in concert with Factor VIIa or a Factor VIIa equivalent. In some embodiments, the additional agent comprises a coagulant, including, without limitation, a coagulation factor such as, e.g. Factor V (see, e.g., PCT/DK02/00736), Factor VIII, Factor IX (see, e.g., WO 02/062376), Factor X, Factor XI, Factor XIII (see, e.g., WO 01/85198), Fibrinogen, thrombin, TAFI (see, e.g., PCT/DK02/00734), Antifibrinolytics such as, e.g., PAI-1, aprotinin, epsilon-aminocaproic acid or tranexamic acid (see, e.g., PCT/DK02/00735; PCT/DK02/00742; PCT/DK02/00751; PCT/DK02/00752); various antithrombotic treatments, as well as transfusions with platelet, RBC, FFP, oxygen carriers, the various bypassing agents and fluid therapies (colloids/crystalloids) or any combination thereof. [Are these also relevant?: inhibitors of tissue factor pathway inhibitor (TFPI inhibitors) (see, e.g., WO 01/85199); protein C inhibitors (see, e.g., PCT/DK02/00737); thrombomodulin (see, e.g., PCT/DK02/00738); protein S inhibitors (see, e.g., PCT/DK02/00739); tissue plasminogen activator inhibitors (see, e.g., PCT/DK02/00740); α2-antiplasmin (see, e.g., PCT/DK02/00741);

It will be understood that, in embodiments comprising administration of combinations of Factor VIIa with other agents, the dosage of Factor VIIa or Factor VIIa equivalent may on its own comprise an effective amount and additional agent(s) may further augment the therapeutic benefit to the patient. Alternatively, the combination of Factor VIIa or equivalent and the second agent may together comprise an effective amount for treating burn trauma. It will also be understood that effective amounts may be defined in the context of particular treatment regimens, including, e.g., timing and number of administrations, modes of administrations, formulations, etc.

### Treatment Outcomes:

The present invention provides methods and compositions for treating burn trauma. Treatment encompasses any measurable improvement or amelioration of any parameter that is indicative of the degree of burn trauma. Non-limiting examples of such parameters include:
Coagulation status, as reflected, e.g. in abnormalities resembling DIC; excessive fibrinolysis; dilutional coagulopathy, including, without limitation, a limited number of platelets and/or an impaired platelet function compared to the platelet count and platelet activity of normal pooled blood
Hypothermia, a including having body temperature below about 37° C., such as, e.g., below about 36° C., below about 35° C., or below about 34° C.
Indicators of metabolic abnormalities, including, without limitation, acidosis having a blood pH below about 7.5, such as, e.g., below about 7.4, below about 7.3, below about 7.2, or below about 7.1.
Blood loss

Efficacy of the methods of the present invention in treating burn trauma may potentially also be measured by assessing a statistical decrease in late complications, including, without limitation, Pulmonary embolism (PE), Acute Respiratory Distress Syndrome (ARDS), Disseminated Intravascular Coagulation (DIC), Acute Myocardial Infarction (AMI), Cerebral Thrombosis (CT), Systemic Inflammatory Response Syndrome (SIRS), infections, Multiple Organ Failure (MOF), and Acute Lung Injury (ALI), including death caused by one or more of these syndromes.

In practicing the present invention, late complications may be assessed using conventional methods, such as, e.g. general coagulation-related parameters such as APTT, Fibrinogen, platelets, D-dimers, Anti thrombin-III, F1+2, TAT, INR, Haematocrit, Haemoglobin and White Blood Cells.

Blood may also be analysis for: S-Bilirubin, S— albumin, S-creatinine, S-potassium, S-sodium, S-alanine aminotransferase, and FVII:C (Pharmacokinetics).

Blood Chemistry. Assessments may be performed at least about 20 days from the start of treatment according to the invention, such as, e.g., at least about 30 days, at least about 35 days, or at least about 40 days from the start of treatment.

Organ damage or organ failure encompass, without limitation, damage to the structure and/or damage to the functioning of the organ in kidney, lung, adrenal, liver, bowel, cardiovascular system, and/or haemostatic system. Examples of organ damage include, but are not limited to, morphological/structural damage and/or damage to the functioning of the organ such as, for example accumulation of proteins (for example surfactant) or fluids due to pulmonary clearance impairment or damage to the pulmonary change mechanisms or alveolo-capillary membrane damage. The terms “organ injury”, “organ damage” and “organ failure” may be used interchangeably. Normally, organ damage results in organ failure. By organ failure is meant a decrease in organ function compared to the mean, normal functioning of a corresponding organ in a normal, healthy person. The organ failure may be a minor decrease in function (e.g., 80-90% of normal) or it may be a major decrease in function (e.g., 10-20% of normal); the decrease may also be a complete failure of organ function. Organ failure includes, without limitation, decreased biological functioning (e.g., urine output), e.g., due to tissue necrosis, loss of glomeruli (kidney), fibrin deposition, haemorrhage, oedema, or inflammation. Organ damage includes, without limitation, tissue necrosis, loss of glomeruli (kidney), fibrin deposition, haemorrhage, oedema, or inflammation.

Lung damage encompasses, but is not limited to, morphological/structural damage and/or damage to the functioning of the lung such as, for example accumulation of proteins (for example surfactant) or fluids due to pulmonary clearance impairment or damage to the pulmonary change mechanisms or alveolo-capillary membrane damage. The terms “lung injury”, “lung damage” and “lung failure” may be used interchangeably.

Methods for testing organ function and efficiency, and suitable biochemical or clinical parameters for such testing, are well known to the skilled clinician.

Such markers, or biochemical parameters of organ function are, for example:

| | |
|---|---|
| Respiration: | PaO2/FiO2 ratio |
| Coagulation: | Platelets |
| Liver: | Bilirubin |
| Cardiovascular: | Blood pressure and need for vasopressor treatment |
| Renal: | Creatinine and urine output |

Other clinical assessments comprise ventilator free days, organ failure free days, vasopressor treatment free days, SOFA score and Lung Injury Score evaluation as well as vital signs.

Methods for testing for coagulophathy or inflammation are also well known to the skilled clinician. Such markers of coagulatory or inflammatory state are, for example, PTT, Fibrinogen depletion, elevation in TAT complexes, ATIII activity, IL-6, IL-8, or TNFR-1.

Chronic organ damage encompasses, but is not limited to, the long-term damage that may result from ARDS. This residual impairment, in particular of pulmonary mechanics, may include, without restriction, mild restriction, obstruction, impairment of the diffusing capacity for carbon monoxide, or gas-exchange abnormalities with exercise, fibrosing alveolitis with persistent hypoxemia, increased alveolar dead space, and a further decrease in alveolar or pulmonary compliance. Pulmonary hypertension, owing to obliteration of the pulmonary-capillary bed, may be severe and lead to right ventricular failure.

In the present context, prevention includes, without limitation, the attenuation, elimination, minimization, alleviation or amelioration of one or more symptoms or conditions associated with late complications associated with burn trauma, including, but not limited to, the prevention of further damage to and/or failure of organs already subject to some degree of organ failure and/or damage, as well as the prevention of damage and/or failure of further organs not yet subject to organ failure and/or damage. Examples of such symptoms or conditions include, but are not limited to, morphological/structural damage and/or damage to the functioning of organs such as, but not limited to, lung, kidney, adrenal, liver, bowel, cardiovascular system, and/or haemostatic system. Exampies of such symptoms or conditions include, but are not limited to, morphological/structural damage and/or damage to the functioning of the organs such as, for example, accumulation of proteins (for example surfactant) or fluids due to pulmonary clearance impairment or damage to the pulmonary exchange mechanisms or damage to the alveolo-capillary membrane, decreased urine output (kidney), tissue necrosis, loss of glomeruli (kidney), fibrin deposition, haemorrhage, oedema, or inflammation.

Attenuation of organ failure or damage encompasses any improvement in organ function as measured by at least one of the well known markers of function of said or gans (see Tables 2 to 5) compared to the corresponding value(s) found in burn trauma patients not being treated in accordance with the present invention.

Prevention also includes preventing the development of Acute Lung Injury (ALI) into ARDS. ALI is defined by the following criteria (Bernard et al., Am. J. Respir. Crit. Care Med 149: 818-24, 1994): acute onset; bilateral infiltrates on chest radiography; pulmonary-artery wedge pressure of ≦18 mm Hg or the absence of clinical evidence of left atrial hypertension; and PaO₂:FiO₂ of ≦300. ARDS is defined by the following criteria (Bernard et al., Am. J. Respir. Crit. Care Med 149: 818-24, 1994): acute onset; bilateral infiltrates on chest radiography; pulmonary-artery wedge pressure of ≦18 mm Hg or the absence of clinical evidence of left atrial hypertension, and PaO₂:FiO₂ of ≦200. (PaO₂ denotes partial pressure of arterial oxygen, and FiO₂ fraction of inspired oxygen).

### Measurement of Late Complications:

Following are non-limiting examples of methods for assessing the incidence and severity of late complications of burn trauma.

TABLE 2
The Glasgow Coma Score is determined as follows
Glasgow Coma Scale

| Eye Opening | | Motor Response |
|---|---|---|
| (E) | Verbal Response (V) | (M) |
| 4 = Spontaneous | 5 = Normal conversation | 6 = Normal |
| 3 = To voice | 4 = Disoriented conversation | 5 = Localizes to pain |
| 2 = To pain | 3 = Words, but not coherent | 4 = Withdraws to |
| 1 = None | 2 = No words . . . only | pain 30 |
| | sounds | 3 = Decorticate |
| | 1 = None | posture |
| | | 2 = Decerebrate |
| | | 1 = None |
| | | Total = E + V + M |

Normal = 15
Vegetative = 0

TABLE 3
The Multiple Organ Failure (MOF) score is determined as follows:
Multiple Organ Failure Score

| | | An MOF score of 4 or more | | |
|---|---|---|---|---|
| Multiple Organ Failure | | Grade 1 | Grade 2 | Grade 3 |
| | Grade 0 | Dysfunction | Dysfunction | Dysfunction |
| Pulmonary^{a} | Normal | ARDS score >5 | ADRS score >9 | ARDS score |
| | | | | >13 |
| Renal | Normal | Creatinine >1.8 mg/dL | Creatinine >2.5 mg/dL | Creatinine >5 mg/dL |
| Hepatic^{b} | Normal | Bilirubin >2 mg/dL | Bilirubin >4 mg/dL | Bilirubin >8 mg/dL |
| Cardiac^{c} | Normal | Minimal inotropes | Moderate inotropes | High inotropes |

^{a}ARDS score = A + B + C + D + E, PCWP ≦ 18 cm H₂0, or clinical setting where high PCWP is not anticipated.
^{b}Biliary obstruction and resolving haematoma excluded.
^{c}Cardiac index <3.0 L/min/m2 requiring inotropic support. Minimal dose, dopamine or dobutamine <5 μg/kg/min; moderate dose, dopamine or dobutamine 5-15 μg/kg/min; high dose, greater than moderate doses of above agents.
Healthy = 0
Severe = 15

TABLE 4
The ARDS Score is determined as follows:
ARDS Score

A. Pulmonary findings by plain chest
radiography
0 = Normal
1 = Diffuse, mild interstitial
marking/opacities
3 = Diffuse, moderate airspace
consolidation
4 = Diffuse, severe airspace
consolidation
B. Hypoxemia - Pao₂/Fio₂ (mmHg)
0 = Normal
1 = 175-250
2 = 125-174
3 = 80-124
4 = <80
C. Minute ventilation (L/min)
0 = <11
1 = <11-13
2 = <14-16
3 = <17-20
4 = >20
D. Positive and expiratory pressure
(cm H₂0)
0 = <6
1 = 6-9
2 = 30-39
3 = 14-17
4 = >17
E. Static compliance (mL/cmH₂0)
0 = >50
1 = 40-50
2 = 30-39
3 = 20-29
4 = <20
Normal = 0
Severe = 20

TABLE 5
The SIRS Score is determined as follows:
Systemic Inflammatory Response Syndrome Score
A SIRS score (1 to 4) calculated for each subject.

One point for each component present:
fever or hypothermia
tachypnea
tachycardia
leukocytosis
SIRS is present when two or more of the following criteria are met:
temperature greater than 38° C. or less than 36° C.
heart rate greater than 90 beats per minute
respiratory rate greater than 20 breaths per minute or PaCO₂ less than 32
white blood cell count greater than 12,000/mm³ or less than 4,000/,mm³ or
presence of 10% bands
Normal = 0
Severe = 4

TABLE 6
DIC is measured as follows:
DIC

| Term: | Definition: |
|---|---|
| Disseminated | Clinical history of: an intense clotting stimulus and |
| Intravascular | shock (infection, trauma, tissue damage, surgery) |
| Coagulation | followed by bleeding. Blood tests: |
| | fibrinogen ≦150 mg/dL |
| | platelet count <150,000//mm³ or drop of 100,000/mm³ |
| | from last valve |
| | D-dimer >500 μg/L |

In one series of embodiments, the practice of the present invention results in one or more of the following clinical outcomes:
A decrease in blood loss, including a complete cessation of blood loss
An improvement in one or more parameters of shock, including, e.g., hypothermia and blood pH.

In one series of embodiments, the practice of the present invention results in one or more of the following clinical outcomes:
A Glasgow Coma Score of greater than about 9 when measured 20 days after start of treatment;
A Glasgow Coma Score of greater than about 11 when measured 30 days after start of treatment;
A Glasgow Coma Score of greater than about 13 when measured 40 days after start of treatment;
An MOF Score of less than about 4 when measured 20 days after start of treatment;
An MOF Score of less than about 3 when measured 30 days after start of treatment;
An MOF Score of less than about 2 when measured 40 days after start of treatment;
An ARDS Score of less than about 8 when measured 20 days after start of treatment;
An ARDS Score of less than about 6 when measured 30 days after start of treatment;
An ARDS Score of less than about 4 when measured 40 days after start of treatment;
An SIRS Score of less than about 3 when measured 20 days after start of treatment;
An SIRS Score of less than about 2 when measured 30 days after start of treatment;
An SIRS Score of less than about 1 when measured 40 days after start of treatment:
Any combination of any of the above Glasgow Coma Scores, MOF Scores, ARDS Scores, and/or SIR Scores.

### Other Indices of Treatment:

The efficacy of the methods of the present invention may also be assessed using other clinical parameters, including, without limitation, reduction in any one or more of the following parameters relative to a similar patient who has not been administered Factor VIIa or a Factor VIIa equivalent according to the invention: a reduction in units of blood, plasma, red blood cells, packed red blood cells, or volume replacement products that need to be administered; a decrease in the number of days of hospitalization after suffering a burn trauma, including a decrease in the number of days that a patient may spend in an intensive care unit (ICU) and a decrease in the number of days in which certain interventions (such as, e.g., a ventilator) are required. Non-limiting examples of outcomes include: (i) a reduction in the units of blood, plasma, red blood cells, packed red blood cells, or volume replacement products that need to be administered by at least about 2 units, 4 units, or 6 units; (ii) a decrease in ICU days by 1 day, 2 days, or 4 days; (iii) a reduction on the number of days on a ventilator by 1 day, 2 days, or 4 days; (iv) a reduction in the total days of hospitalization by 2 days, 4 days, or 8 days.

The present invention is further illustrated by the following examples which, however, are not to be construed as limiting the scope of protection. The features disclosed in the foregoing description and in the following examples may, both separately and in any combination thereof, be material for realising the invention in diverse forms thereof.

### BRIEF DESCRIPTION OF THE DRAWINGS

FIG. 1. The levels of FVII:C in the rFVIIa (full square) and placebo (empty square) groups. Each data point represents the mean and SEM of 6-8 patients.

FIG. 2. The levels of thrombin-antithrombin complex in the rFVIIa (full square) and placebo (empty square) groups. Each data point represents the mean and SEM of 6-8 patients.

FIG. 3. The levels of interleukin-6 in the rFVIIa (full square) and placebo (empty square) groups. Each data point represents the mean and SEM of 6-8 patients.

### EXAMPLES

### Example 1

The inventors of the present invention have investigated the pro-hemostatic effect and safety of a Factor VIIa or a corresponding amount of a Factor VIIa equivalent, such as NovoSeven® in (i) a standardised cohort of patients, were bleeding were a major clinical problem, (ii) patients not having concurrent illnesses or medications that could interfere with the study result interpretation, (iii) patients treated in a standardised way, with regard to surgery, anaesthesia, transfusion practice, postoperative treatment and rehabilitation.

### Factor VIIa Administration to Burn Trauma Victims

The following study was performed in order to assess efficacy and safety of recombinant activated coagulation factor VII (rFVIIa, NovoSeven®) as adjunctive therapy for bleeding control in severe burn trauma.

We have conducted a placebo-controlled, randomized study in 18 patients with burn injuries requiring skin grafting of more than 10% of the TBSA. A dose of 80 μg/kg rFVIIa were administered immediately prior to surgery and repeated 1 hour thereafter. In our study, there were no clinical signs or symptoms of thromboembolic complications recorded during the study period (30 days). Furthermore, administration of rFVIIa significantly reduces 24-hour postoperative transfusion requirements, compared to the placebo group p<0.05. These findings supports the concept that rFVIIa initiates a localised thrombin generation at site of vascular damage, ultimately leading to fibrin polymerisation and achievement of haemostasis, not affecting the systemic coagulation of the patient.

### Study Design I:

A prospective double blind, randomized placebo controlled study of NovoSeven® on per and 24-hour postoperative transfusion requirements.

Patients with a burn injury, needing an excision of 10% or more of TBSA were included. Patients were excluded if they had: 1) inhalation injury, 2) a pre-existing inherited or acquired coagulopathy, 3) significant liver or kidney impairment, 4) E F below 40% or AMI within 3 months of surgery, 5) any thrombo-embolic condition within 3 months of surgery.

Sepsis, needing inotropic support of more than 4 μg/ml dopamine/hour.

### Study Design II:

Surgery was performed by the same 2 surgeons in all patients

The patients were anaesthesised by the same 2 anaestesiologists, and received the same type of anaesthesia.

Blood samples were drawn at T=0, and 20, 40, 60, 120, 240, and 480 minutes thereafter.

Analysis of Hb, Hct, Platelets, aPTT, INR, F1+F2, D-dimer was performed every 12 hours the first 7 days postoperatively.

Physical examination was performed twice daily, the first 7 days postoperatively

All patients received postoperative antithrombotic prophylaxis.

### Method:

Recombinant human FVIIa 80 μg/kg or Placebo was given immediately before start of surgery and repeated 60 minutes thereafter.

Tangential excision down to viable tissue, not involving the muscular fascia, was used.

Donor harvest was performed before excision of the burn injury.

Transfusion of RBC when hematocrit reached 25%, FFP was transfused in a 1:1 ratio with the RBC transfused and PLT was transfused after every 8 RBC.

### Assessment:

Microvascular bleeding (MVB) was defined as:

1. Profuse, oozing type of bleeding from the surgical field, and/or
2. Start of bleeding from areas were haemostasis previously had been achieved, and/or
3. Beginning of bleeding from intra vascular access lines (CVK, arterial and venous catheters).
4. Together with signs of systemic hypotension and/or increased transfusion requirements.

MVB was assessed continuously.

### End Points:

### Primary:

1. Per and 24-hour postoperative RBC transfusion requirements.
2. Total per and 24-hour postoperative transfusion requirements (RBC+FFP+PLT).

### Secondary:

1. Development of per-operative micro-vascular bleeding.
2. 30-day survival.
3. Adverse events; especially thrombo-embolic complications during the 30-day study period.

### Results:

No differences between treatment groups with regard to age, gender, surgical time, hospital stay or mean TBSA excised, which was 19% in the Placebo group and 22% in the Recombinant human FVIIa group.

### Transfusion RBC Requirements:

rFVIIa group: mean 5 units, SD 5.78.
Placebo group: mean 15 units, SD 11.02 p=0.035.
Total transfusion requirements (RBC+FFP+PLT):
rFVIIa group: mean 17 units, SD 20.43.
Placebo group: mean 36 units, SD 22.52 p=0.041.

Patients developing MVB had significantly larger TBSA % excised, 29% compared to 18% in the non-MVB group, p=0.03.

Patients developing MVB had significantly larger RBC transfusion requirement; 21 units compared to 7 units in the non-MVB group, p=0.005.

Total transfusion requirements in the MVB group was 50 units compared to 20 units in the non-MVB group, p=0.007.

Survival was 1 of 4 patients in the MVB group, compared with 14 of 14 in the rest of the cohort, p=0.005.

30 day survival was 15/18 patients or 83%.

All non-surviving patients (3) were from the Placebo group, p=0.10 (Fischer exact test). Micro-vascular bleeding occurred in 4 patients, 3 in the Placebo group, and 1 in the rFVIIa group, p=0.3.

There were no adverse events, and especially no thrombo-embolic complications.

Patients developing MVB had significantly larger TBSA % excised, 29% compared to 18% in the non-MVB group, p=0.03.

Patients developing MVB had significantly larger RBC transfusion requirement; 21 units compared to 7 units in the non-MVB group, p=0.005.

Total transfusion requirements in the MVB group was 50 units compared to 20 units in the non-MVB group, p=0.007.

Survival was 1 of 4 patients in the MVB group, compared with 14 of 14 in the rest of the cohort, p=0.005.

### Conclusions:

rFVIIa; Recombinant human FVIIa at a dose of 80 μg/kg×2 significantly reduces per and 24-hour postoperative transfusion requirements in patients with burn injury of 10% or more excised and skingrafted

rFVIIa; NovoSeven at a dose of 80 μg/kg×2, is safe in patients with burn injury, undergoing tangential excision and skin grafting

Burn patients developing MVB per-operatively have a significantly higher transfusion requirement and a lower 30-day survival, than patients not developing MVB.

### Implications:

The study showed better survival, and lower frequencies of development of micro-vascular bleeding, in patients receiving rFVIIa; NovoSeven.

The safety of using rFVIIa; NovoSeven in this category of patients, and the current knowledge of mechanism of action, implies that it would be safe to use rFVIIa; NovoSeven, also in other patient categories with high TF expression (Trauma, Sepsis, DIC, ICH)

### Example 2

Eighteen consecutive patients scheduled for the surgery were randomised to receive either placebo or 40 μg/kg rFVIIa administered at first skin incision, and a second dose (40 μg/kg) at 90 minutes later. Blood transfusion requirements during, and up to 24 hours post-surgery were compared. In addition, postoperative complications commonly seen in patients with burn injury as well as adverse events related to rFVIIa were monitored.

rFVIIa significantly decreased the total number of units of blood components transfused compared with placebo (17 vs 37, p=0.01). We further observed a trend towards improved graft survival (p=0.1) and reduction in multiple organ failures (p=0.08) in the rFVIIa-treated group. There were no adverse events, in particular thrombo-embolic events, considered to be drug related.

### Study Design:

The study was a single-centre, randomised, double-blind, placebo-controlled trial conducted at the University Hospital of Copenhagen. The trial protocol was approved by the local Institutional Ethics Committee and written informed patient consent was obtained.

The inclusion criteria were: patients with thermal burn aged ≧18 years who were scheduled to have full thickness burn wound excision of more than 10% of the total body surface area (estimated by Rule of Nines) and skin grafting. The exclusion criteria were: patients with contraindication for postoperative thromboprophylaxis with low-molecular-weight heparin (LMWH); patients who had received non-steroidal anti-inflammatory drug (NSAID) within seven days prior to the surgery; patients with sepsis, human immunodeficiency virus (HIV) positive, pregnancy, creatinine clearance less than 25 mL/min, advanced liver cirrhosis or acute hepatitis, renal failure requiring dialysis, known coagulopathy, known severe atherosclerosis (history of acute myocardial infarction, diabetes mellitus, severe hypertension), or history of deep vein thrombosis within the last six months.

Patients who met the inclusion criteria were randomised, at the time of screening, to receive either placebo or rFVIIa (NovoSeven®, Novo Nordisk A/S, Bagsvaerd, Denmark) 40 μg/kg given as an intravenous bolus injection immediately before the start of surgery, and a second dose (40 μg/kg) was given at 90 minutes later. Tangential excision technique was used. All surgical procedures were carried out by the same surgical team. After surgery, all patients received thromboprophylaxis with LMWH until they were fully mobile or discharged.

Perioperative (during and up to 24 hours after surgery) blood transfusion was decided by an anaesthetist who was blinded to the treatment. The following transfusion guidelines were followed: red blood cells (RBC) were transfused when a haemoglobin (Hb) concentration dropped below 5.5 mmol/L. Fresh frozen plasma (FFP) was transfused at a 1:1 ratio to RBC, or there were signs of microvascular bleeding. Platelet concentrates were given when platelet count <80×109/L, or for every estimated blood volume transfused, or when there were signs of microvascular bleeding. Microvascular bleeding was defined as re-bleeding from skin donor sites where haemostasis was previously achieved together with a decrease in systemic blood pressure. All blood components used were leucodepleted and no RBC units older than 7 days were transfused. The primary endpoint was the total number of units of blood components transfused (RBC+FFP+platelets) during and up to 24 hours after surgery. The secondary endpoints were the operating time, the number of patients with microvascular bleeding, % graft survival on day 7 after surgery, days spent in intensive care unit (ICU) after surgery, days of hospitalisation, and patient survival rate on day 30 after surgery. Furthermore, postoperative complications commonly seen in patients with burn injury were recorded until patients were discharged from the hospital. These included wound infection, pneumonia, sepsis, acute lung injury, renal failure, circulatory failure, and multiple organ failure. In addition, all patients were monitored for adverse events related to the trial drug, in particular thromboembolic events, for 30 days.

Haemoglobin concentration, platelet count, and prothrombin time-international normalised ratio (PT-INR) were measured immediately before the start of surgery, and at 2 and 4 hours later. In addition, factor VII:clotting activity (FVII:C), thrombin-antithrombin complex (TAT; Enzygnost TAT micro, Dade Behring), tissue factor (TF; Imubind TF ELISA kit, American Diagnostica Inc.), and interleukin-6 (IL-6; Quantikine Human IL-6 Immunoassay, R&D Systems) were measured immediately prior to start of surgery, and thereafter at 20 min, 40 min, 1 hr, 2 hr, 4 hr, and 8 hr. All assays were performed by laboratory technicians who were blinded to the treatment.

Results from the rFVIIa and placebo groups were compared using the unpaired ttest, two-sample Wilcoxon rank sum test or Fisher's exact test for count data as appropriate. A p-value ≦0.05 was considered statistically significant.

### Results:

21 consecutive patients met the inclusion criteria and 18 were included in the study. Three patients were excluded because neither the surgeon nor the anaesthetists were available. Nine patients received rFVIIa and nine received placebo. There were no statistically significant differences in the baseline characteristics between patients in the placebo and the rFVIIa groups (Table 7). All patients underwent one-stage surgery 4 days or later after the burn injury.

Results are summarised in Table 8. The total number of units of blood transfused (RBC+FFP+platelets) was significantly reduced in the rFVIIa group compared with the placebo group (median 17 vs 37, p=0.01). One patient in the rFVIIa group and four in the placebo developed microvascular bleeding (p=0.29). These patients required significantly more blood transfusions than those who did not develop microvascular bleeding (37 vs 18, p<0.01). Moreover, in the rFVIIa group, there was a trend toward a reduction in development of multiple organ failure (p=0.08), and towards an increase in the percentage of graft survival (p=0.1), compared with the placebo group.

Concerning the number of patients who developed postoperative sepsis, wound infection or pneumonia, there was no difference between the rFVIIa and placebo groups. Three patients did not survive the 30 day study period; they were all from the placebo group. There were no adverse events considered related to rFVIIa.

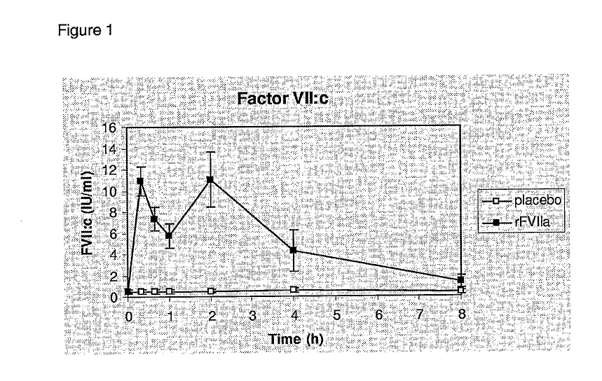

The preoperative PT-INR values were within the normal range in both groups of patients. The administration of rFVIIa shortened the median PT-INR from 1.20 before treatment to <0.70 and began to return to baseline values at 8 hours (data not shown). The PT-INR in the placebo group remained unchanged. The concentrations of FVII:C were within normal range (0.54-1.23 U/mL) in both the rFVIIa and placebo groups before start of surgery. The level remained unchanged in the placebo group, whereas in the rFVIIa group, FVII:C increased by 20-fold after dosing (FIG. 1).

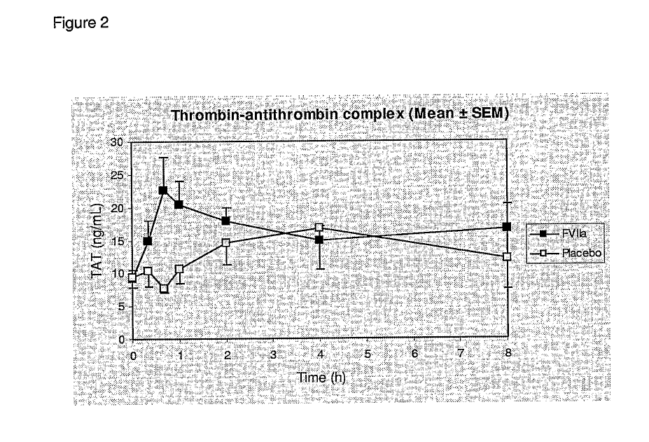

The baseline levels of TAT were similar in both groups, and were higher than the normal range (1.0-4.1 ng/mL) (FIG. 2). Approximately 40 minutes after the administration of rFVIIa, the levels of TAT was significantly higher than those in the placebo group (p=0.05) but this effect abated within one hour after the administration of the second dose.

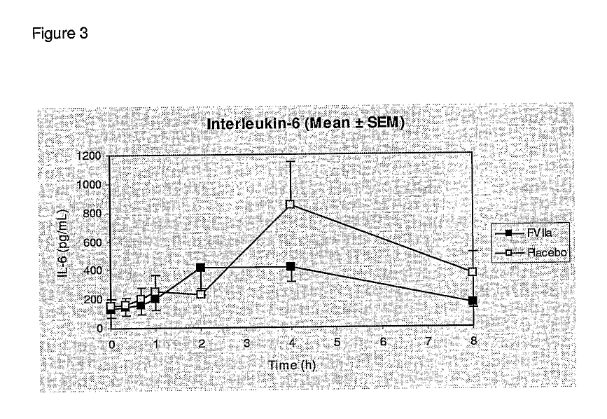

The plasma IL-6 concentrations in all patients before surgery were higher than normal range (<3.0 pg/mL) (FIG. 3). Interestingly, in the placebo group, surgery induced a significant (p=0.05), 6-fold increase in plasma IL-6 levels, reaching maximum levels at 4 hours after start of surgery. In contrast, in the rFVIIa treated patients, the peak levels of IL-6 were reached somewhat earlier at 2-4 hours and surgery only induced a non-significant 3.3-fold increase in IL-6 levels in this group. These findings indicate that rFVIIa modulated the proinflammatory cytokine response in these patients.

The plasma TF concentrations were not statistically different between groups and were within the normal range (149±72 pg/mL) at all measured time points (data not shown).

TABLE 7
Patient characteristics

| | Placebo (n = 9) | rFVIIa (n = 9) |
|---|---|---|
| Gender | | |
| Male | 6 | 6 |
| Female | 3 | 3 |
| Age (years) | 38 (19-81) | 54 (22-85) |
| Total burn surface area (%) | 20 (15-60) | 25 (12-53) |
| Total surgical area (%) | 17 (11-36) | 19 (12-45) |
| Haemoglobin (mmol/L) | 6.0 (5.5-6.6) | 6.1 (5.3-6.4) |
| Platelet count | 281 (89-705) | 268 (110-653) |
| PT (INR) | 1.2 (1.1-1.8) | 1.2 (1.1-1.6) |
| Time from burn to surgery (days) | 7 (4-16) | 10 (4-17) |

Data are shown as median (range) when applicable.

TABLE 8
Trial results

| | Placebo | rFVIIa | p |
|---|---|---|---|
| | (n = 9) | (n = 9) | value* |
| Total number of units of blood | 37 (15-84) | 17 (14-35) | 0.01 |
| transfused | | | |
| Operating time (minutes) | 140 (95-200) | 120 (65-190) | 0.76 |
| Number of patients with | 4 | 1 | 0.29 |
| microvascular bleeding | | | |
| % Graft survival on postop | 75 (65-100) | 95 (65-100) | 0.10 |
| day 7 | | | |
| Patient survival rate on postop | 66.7% | 100% | 0.20 |
| day 30 | | | |
| Time in ICU (days) | 8 (0-37) | 4 (0-63) | 0.59 |
| Time in hospital (days) | 36 (28-72)^{†} | 49 (33-110)^{‡} | 0.22 |
| Postoperative complications: | | | |
| Wound infection | 2 | 2 | 0.71 |
| Sepsis | 6 | 5 | 0.50 |
| Pneumonia | 5 | 6 | 0.50 |
| Acute lung injury | 1 | 2 | 0.50 |
| Multiple organ failure | 7 | 3 | 0.08 |

Data are shown in median (range) when applicable.
*Two-sample Wilcoxon rank sum test or Fisher's exact test for count data as appropriate.
^{†}Three patients died on days 7, 20 and 20; and were excluded.
^{‡}One patient died on day 63, and was excluded.

The present example is another study on the efficacy and safety of rFVIIa in patients with full thickness burn injury undergoing excision and skin grafting. The empirical dose regimen used in this study (40 μg/kg for two doses) significantly decreased the overall blood transfusion requirement. Patients receiving rFVIIa had an increased survival rate compared to the placebo group (9 of 9 vs. 6 of 9) and there was a trend towards a decrease in the number of patients who developed multiple organ failure. Furthermore, a trend towards increased graft survival in the rFVIIa treated group as compared to the placebo group was observed, indicating that rFVIIa treatment does not compromise the local homeostasis in the affected tissues. Four patients in the placebo group developed microvascular bleeding as compared to only one patient in the rFVIIa group. These patients required significantly more transfusions as compared to the rest of the cohort. The patient in the rFVIIa group who developed microvascular bleeding did so 80 minutes after the first dose of rFVIIa was administered and excision of 35% of total body surface area and skin harvest from donor site had been completed. Five minutes after the administration of the second dose of rFVIIa, the microvascular bleeding ceased and did not reappear, showing the pro-haemostatic effect of rFVIIa in this bleeding condition which is usually difficult to treat.

The increased levels of TAT before start of surgery confirm the hypercoagulable state which is a characteristic of these patients. Administration of rFVIIa significantly increased the levels of TAT further, providing an evidence of rFVIIa-enhanced thrombin generation. Both FVII:C and TAT concentrations approached the baseline values 8 hours after the administration of the first does. There were no thromboembolic adverse events in this study.

In patients with burn injury, suppression of cellular immune function resulting in increased susceptibility to bacterial infection is a major complication which directly affects morbidity and mortality. The macrophages and the production of cytokines, including IL-6, a pleiotropic cytokine involved in the regulation of the immune and acute phase responses, play a central role in triggering immune dysfunction after burn and traumatic injury. Several clinical conditions such as burn, trauma, cancer, and infection have been associated with abnormally high levels of circulating IL-6 and abnormal immune responses. High levels of IL-6 suppress T cell function and correlate positively with increased mortality. Furthermore, in critically ill patients, the interaction between the activation of the coagulation system and the inflammatory response may worsen the clinical outcome, resulting in multiple organ failure and ultimately mortality.

In this study, all patients had elevated IL-6 levels prior to surgery reflecting the inflammatory response to injury. The surgical procedure resulted in a further, transient elevation of the IL-6 levels, reaching its highest levels four hours after the start of surgery in the placebo-treated patients. Interestingly, in patients receiving rFVIIa, the surgery-induced IL-6 was not significantly elevated compared to the pre-surgical baseline and reached the peak earlier, at 2-4 hours after the start of surgery. This finding suggests that rFVIIa may modulate the inflammatory response to surgery-induced tissue injury. We speculate that this inflammatory modulating effect may, at least in part, explain the observed trend towards fewer multiple organ failures in the rFVIIa-treated group. From these findings, we suggest, for the first time, that the use of rFVIIa, by a mechanism currently unknown, in addition to its haemostatic potential may benefit this patient population by reducing surgery-triggered inflammation. In contrast to a previous report, we did not observe a concomitant increase in circulating TF levels despite the increased IL-6 levels.

1-10. (canceled)

11. Kit of parts for treatment of burn trauma, comprising
(i) A medicament comprising Factor VIIa or a Factor VIIa equivalent; and
(ii) Instructions for Use describing that:
a. A first dose containing at least about 40 μg/kg Factor VIIa or a corresponding amount of a Factor VIIa equivalent should be administered at the start of treatment;
b. A second dose containing at least about 40 μg/kg Factor VIIa or a corresponding amount of a Factor VIIa equivalent should be administered one to 24 hours after the start of treatment.

12. Kit according to claim 11, wherein the instructions for use further describes that an optional third dose containing at least about 40 μg/kg Factor VIIa or a corresponding amount of a Factor VIIa equivalent may be administered to said patient at least about one hour after the start of the second treatment.

13. A method for treating burn trauma, the method comprising administering to a patient in need of said treatment an effective amount for said treatment of Factor VIIa or a Factor VIIa equivalent.

14. A method according to claim 13, wherein the patient is suffering from burn trauma needing an excision of tissue of 10% or more of total body surface area (TBSA).

15. A method according to claim 13, wherein the patient has microvascular bleedings.

16. A method according to claim 13, wherein said effective amount comprises at least about 40 μg/kg Factor VIIa or a corresponding amount of a Factor VIIa equivalent.

17. A method according to claim 13, wherein a first amount of at least about 40 μg/kg Factor VIIa or a corresponding amount of a Factor VIIa equivalent is administered at the start of treatment, and a second amount of at least about 40 μg/kg Factor VIIa or a corresponding amount of a Factor VIIa equivalent is administered to the patient one to 24 hours after the start of treatment.

18. A method according to claim 17, further comprising administering to the patient a third amount of at least about 40 μg/kg Factor VIIa or a corresponding Factor VIIa equivalent at least about one hour after the start of the second treatment.

19. A method according to claim 13, further comprising administering to the patient a second coagulation agent in an amount that augments said treating by said Factor VIIa or Factor VIIa equivalent.

20. A method according to claim 19, wherein said second coagulation agent is selected from the group consisting of a coagulation factor and an antifibrinolytic agent.

21. A method according to claim 20, wherein said coagulation agent is selected from the group consisting of Factor V, Factor VIII, Factor IX, Factor X, Factor XI, Factor XIII, Fibrinogen, thrombin, TAFI, PAI-1, aprotinin, epsilon-aminocaproic acid or tranexamic acid, antithrombotic treatments, transfusions with one or more of platelets, RBCs, FFP, oxygen carriers, the various bypassing agents and fluid therapies (colloids/crystalloids).

22. A method for preventing treating burn trauma, the method comprising intentionally administering to a patient in need of said treatment an effective amount for said treatment of Factor VIIa or a Factor VIIa equivalent for the purpose of treating burn trauma.

23. A method for treating burn trauma in a majority of burn trauma patients, said method comprising (i) administering to a group of burn trauma patients an effective amount for said treatment of Factor VIIa or a Factor VIIa equivalent; and (ii) observing a reduction in one or more clinical parameters of burn trauma among said group of patients relative to the level of said clinical parameters that would have been expected in the same group of patients who had not received said Factor VIIa or Factor VIIa equivalent.
